(19) United States
(12) Patent Application Publication
(10) Pub. No.: US 2014/0196412 A1
(43) Pub. Date: Jul. 17, 2014

(54) MERCAPTAN ABSORBER FOR USE WITH FOOD

(71) Applicant: MULTISORB TECHNOLOGIES, INC., Buffalo, NY (US)

(72) Inventor: George E. McKedy, Williamsville, NY (US)

(73) Assignee: Multisorb Technologies, Inc., Buffalo, NY (US)

(21) Appl. No.: 13/744,003

(22) Filed: Jan. 17, 2013

(51) Int. Cl. A61L 9/01; A61L 9/012; B65B 17/00; A61L 9/014

(52) U.S. Cl. CPC A61L 9/01; A61L 9/014; A61L 9/012; B65B 17/00
USPC 53/474; 423/604; 423/421; 423/599; 423/584; 423/583; 423/493; 423/415.2; 502/416; 423/625; 502/80; 423/700; 252/192

(57) ABSTRACT

The invention provides a food decay odor controller which includes a mercaptan remover.

### FIELD OF THE INVENTION

This invention relates to the improvement of shelf life of foods such as cabbage, garlic and onions by utilization of a mercaptan absorber in packaging.

### BACKGROUND OF THE INVENTION

It is been known to improve the shelf life of some materials by the utilization of oxygen absorbers and water absorbers. Many of these materials comprise iron particles with an acidifier and an electrolyte. Typical of oxygen absorbing materials are those disclosed in U.S. Pat. No. 5,667,863—Cullen et al. and U.S. Pat. No. 6,899,822—McKedy. It is also known to package foods and medical materials with a water vapor absorber. Water vapor absorbers are disclosed in U.S. Pat. No. 7,593,278—Powers et al. and U.S. Pat. No. 6,540,937—Payne et al.

These products have been successful in packaging of many food products and aiding in the preservation of these products to provide a longer shelf life. There remains a need for packaging that will prolong the shelf life of materials that give off mercaptans (also known as thiols) after they are packaged. Materials including onions, garlic, and cabbage give off mercaptans. (Some nuts and cheese along with asparagus contain methyl mercaptan, coffee will also give off mercaptans, methyl mercaptan is one of the main chemicals responsible for bad breath and the odor of flatulence.)

### BRIEF SUMMARY OF THE INVENTION

The invention provides a food decay odor controller which includes a mercaptan remover. In another embodiment, the invention provides for increasing the shelf life of food products which includes packaging the food products with a food decay odor controller including a mercaptan remover.

### DETAILED DESCRIPTION OF THE INVENTION

The invention provides numerous advantages in the packaging of materials that give off mercaptans during storage. The odor removers of the invention allow for longer shelf life of many vegetables. The packages are low-cost and do not provide any exposure of the customer or the vegetable to any hazardous absorbing materials. The mercaptan absorbers may be placed into the packages in a variety of ways to meet the needs of different packaging techniques and different vegetables. These and other advantages of the invention will be apparent from the description below. Based on the work done with the hexanal adsorbers we proved that if activated carbon was used either as a carrier or as an adsorber we could make a combination odor/flavor releaser and hexanal adsorber or a combination ethyl alcohol emitter and hexanal adsorber. This will also work for a mercaptan adsorber. This would also work if molecular sieve or silica gel is used as a carrier or adsorber.

The mercaptans as released by members of the cabbage family such as brussell sprouts, cabbage and kohlrabi, and by onions and garlic are detectable by the typical human nose in very small amounts such as in 1-10 parts per billion of air. The odor is unpleasant and is a rotting smell. These materials give off odor long before their quality as a tasteful and nutritious food has deteriorated. However, consumers find the odor off-putting and will not investigate the vegetable to see if it is still tasty. Therefore, typically many of these materials are not packaged but are placed in bulk on shelves. However, particularly for materials such as chopped cabbage and sliced onions, it would be desirable if they could be packaged in a way that would not allow a buildup of mercaptans in the package to an amount of greater than about 10 ppb or preferably 5 ppb or lower (less than 1 ppb would be best). These mercaptans are primarily believed to be allyl mercaptans.

The odor controller's for food decay that give off mercaptans may be materials that react with the mercaptans or they may be materials that absorb the mercaptans. The odor controllers of the invention are able to reduce airborne mercaptans from a range of 550 ppb in a package to less than 10 ppb, and preferably less than 5 ppb, in order to provide a product that the consumer does not find offensive.

Those materials that will absorb mercaptans and reduce their content in the food package to below the level that can be smelled by the average human include activated carbon, activated alumina, activated carbon with water, and clay impregnated with a sodium hydroxide solution, molecular sieve material and natural zeolites such as chabazite and clinoptilolite.

Odor removers that are able to react with mercaptans and reduce their content to below 10 ppb include sodium permanganate, potassium permanganate, copper chloride, copper oxide hydrogen peroxide, calcium peroxide, sodium chlorite, sodium percarbonate, potassium carbonate, potassium perchlorate, and sodium carbonate. A preferred material has been found to be potassium carbonate as it is very effective in reacting with the mercaptans such that only a small amount of material is necessary. The potassium carbonate worked well but so did sodium permanganate, potassium permanganate, copper oxide, copper chloride, calcium peroxide, hydrogen peroxide, sodium chlorite, and sodium percarbonate.

The sodium permanganate and potassium permanganate oxidize the mercaptan. The copper chloride is believed to react with the mercaptan to form mercaptides. Copper oxide may form a copper complex with the mercaptan. Copper sulfate, manganese sulfate, and other metal oxides also react with mercaptans. Peroxides, such as hydrogen peroxide and calcium peroxide, oxidize the mercaptans. Sodium chlorite with water generates chlorine dioxide which oxidizes the mercaptans. Sodium percarbonate with water generates hydrogen peroxide which oxidizes the mercaptans. Potassium carbonate reacts with the mercaptans forming mercaptides.

In order to be effective in a commercial environment the mercaptan removers must be provided in a package in a form that allows absorption of mercaptans or reaction of mercaptans without interfering with the presentation and use of the vegetable material that is in the package.

Many vegetables are packaged in polyolefin or polyester pouches or bags. These bags generally do not pass odors readily. Therefore, if mercaptan absorbers are placed in the bag they will absorb odors prior to the day the bag is opened and the consumer will not be offended by any rotten (mercaptan) smell. The vegetables also could be packaged in trays that have an adhesive cover of a film, such as polyester. These packages could be provided with a label that contained a layer of odor remover comprising one or more of the above materials. Reference is made to U.S. Pat. No. 5,667,863—Cullen where an oxygen absorber is in a label. In similar manner an odor remover for mercaptans could be placed in a layer of a gas permeable label or a card.

It is also known to place absorbent materials in sachets, in food packages to remove unwanted gases such as oxygen and water vapor. These sachets typically are formed of a gas permeable fabric that is liquid water impermeable. Container for absorbent materials also may be formed as a container in which one side is permeable to gases.

It is also known to place oxygen gas remover in one layer of a film that is adhered to the package in a manner such that the film stays on the package when the vegetable is removed. Such films are similar or identical to label materials, but may be placed in less visible areas such as behind where the containers printed or on the bottom where they are not particularly visible. Films containing mercaptan removers could be placed in vegetable packages.

The film or label materials that may contain the mercaptan remover of the invention may be formed of any suitable polymer. The polymer needs to be safe to consumers and permeable to gases such that the mercaptans can pass into the film and be absorbed or chemically combined. Typically such polymers are polyesters, polyolefins, polyamide, and polymers formed from lactic acid. Preferred polymer materials for incorporating the mercaptans removers comprises low density polyethylene and polypropylene because these are effective and low cost common packaging materials. A preferred mercaptan remover for use in a film of the above polymer is potassium carbonate because it is very reactive with mercaptans. Potassium carbonate is an effective mercaptan absorber and we want an absorber that can be reduced in particle size so that it can be put into the polymer and made into a film.

The following Examples are illustrative and not exhaustive of any of practicing the invention. Parts and percentages are by weight unless indicated otherwise.

### EXAMPLE 1

A gas impermeable polyester bag is filled with 100 grams of sliced Vidalia onion. A sachet of water impermeable and gas permeable spun bond polyolefin containing 1 gram of a 50% solution of potassium carbonate solution absorbed onto filter paper is placed in the bag. The bag is sealed for 10 days. The bag is the probed to determine the concentration of mercaptans. It is found to contain less than 5 ppm of mercaptans and did not exhibit a mercaptan odor. The onion was still tasty.

### EXAMPLE 2

An 11″×16″ foil laminated pouch is vacuumed and then flushed with a gas containing one part per million of allyl mercaptan in nitrogen. The pouch after flushing contains 552-600 ppb of allyl mercaptan by flushing with the 1 ppm of allyl mercaptan in nitrogen. The absorber in the test pouch was 0.5 grams of a 50% solution of potassium carbonate in water (0.25 grams of potassium carbonate and 0.25 grams of water). The mercaptan absorber was measured by using a blank foil pouch with six pieces of 1″×1.5″ bladder paper soaked in saturated magnesium nitrate solution placed in the pouch to act as a humectant. The pouch was flushed with the mercaptan as set forth above and was sealed. After 6 days the pouch was probed and found to contain 1 ppb of allyl mercaptan gas. This demonstrates that the potassium carbonate is effective at removing the mercaptans.

1-26. (canceled)

27. An article comprising:
a package defining an enclosed space;
a food product that gives off mercaptans disposed in the enclosed space; and
a mercaptan controller in communication with the enclosed space and reducing airborne mercaptans in the space to fewer than 10 parts-per-billion.

28. The article of claim 27, wherein the mercaptan controller at least one of absorbs or oxidizes the airborne mercaptans in the space.

29. The article of claim 27, wherein the mercaptan controller comprises at least one of copper oxide, potassium carbonate, sodium permanganate, or potassium permanganate.

30. The article of claim 27, wherein the mercaptan controller comprises at least one of hydrogen peroxide, calcium peroxide, copper chloride, sodium chlorite, or sodium percarbonate.

31. The article of claim 27, wherein the mercaptan controller comprises activated carbon or activated alumina.

32. The article of claim 27, wherein the mercaptan controller comprises at least one of clay, molecular sieve or a natural zeolite.

33. The article of claim 32, wherein the mercaptan controller comprises a natural zeolite and the natural zeolite is chabazite or clinoptilolite.

34. The article of claim 27, wherein the mercaptan controller is impregnated into a polymer film.

35. The article of claim 27, wherein the mercaptan controller includes a clay carrier.

36. The article of claim 27, wherein the mercaptan controller is disposed in a water impervious and gas permeable container disposed in the enclosed space of the package.

37. The article of claim 27, wherein, in the absence of the mercaptan absorber, the airborne mercaptans are present in the head space in an amount of about 550 parts per billion.

38. An article comprising:
a package defining an enclosed space;
a food product comprising at least one of cabbage, brussels sprouts, kohlrabi, onions or garlic disposed in the space; and
a mercaptan controller in communication with the space to at least one of absorb mercaptans or reacts with mercaptans given off by the food product.

39. The article of claim 38, wherein the food product is sliced or chopped prior to being disposed in the space.

40. The article of claim 38, wherein the mercaptan controller absorbs mercaptans and comprises at least one of activated carbon, activated alumina, clay impregnated with a sodium hydroxide solution, molecular sieve, or a natural zeolite.

41. The article of claim 38, wherein the mercaptan controller reacts with mercaptans and comprises potassium carbonate.

42. The article of claim 38, wherein the mercaptan controller reacts with mercaptans and comprises at least one of sodium permanganate, potassium permanganate, copper chloride, copper oxide, hydrogen peroxide, calcium peroxide, sodium chlorite, sodium percarbonate, potassium perchlorate, and sodium carbonate.

43. A method comprising:
placing a food product that gives off mercaptans in a package;
providing a mercaptan controller in communication with a space surrounding the food product to at least one of absorb or oxidize the mercaptans given off by the food product; and
sealing the package,
wherein the odor controller reduces mercaptans in the enclosed space to fewer than 10 parts-per-billion.

44. The method of claim 43, wherein providing the mercaptan controller comprises adhering a carrier carrying the mercaptan controller to the package.

45. The method of claim 43, wherein the food product comprises at least one of cabbage, brussels sprouts, kohlrabi, onions, or garlic.

46. The method of claim 45, wherein the food product is one of chopped or shredded.
